(19) United States
(12) Patent Application Publication
(10) Pub. No.: US 2022/0273272 A1
(43) Pub. Date: Sep. 1, 2022

(54) STABILIZED SALIVA TEST SYSTEM AND METHOD

(71) Applicant: ORAL GENOME CORP., Carlsbad, CA (US)

(72) Inventors: Donna Marie B. Hongo, Vacaville, CA (US); Thanh Luu, Carlsbad, CA (US); Tina Saw, Carlsbad, CA (US); Susan Mahler Zneimer, Camas, WA (US)

(21) Appl. No.: 17/588,607

(22) Filed: Jan. 31, 2022

Related U.S. Application Data
(60) Provisional application No. 63/154,085, filed on Feb. 26, 2021.

(51) Int. Cl. A61B 10/00

(52) U.S. Cl. CPC A61B 10/0051

(57) ABSTRACT

A stabilized saliva testing system and method facilitates obtaining and preparing a stabilized saliva test sample for subsequent analysis. The system preferably includes a kit having various collection and preparation implements, 1× TE pH 8.0 as the stabilizer, a means for mailing the sample, and instructions for use. A user obtains a saliva sample, prepares the stabilized saliva test sample as instructed, and mails the sample to a 3^{rd} party for analysis. The 3^{rd} party tests the sample for tooth and gum health indicators using known testing instrumentation, and sends the user a report summarizing their tooth and gum health.

### BACKGROUND OF THE INVENTION

The present invention relates to testing biological fluids, and more specifically to a system and method of testing stabilized saliva.

It is well established that oral health is extremely important. Oral infections such as tooth decay and gum disease can be unpleasant, unsightly and expensive. What is less known, however, is that there is a synergic relationship between oral health and general health. Gum disease, for example, is linked to a variety of illnesses including heart disease, diabetes, respiratory disease, osteoporosis, and rheumatoid arthritis. People with gum disease are twice as likely as others to die from a heart attack and three times as likely to have a stroke.

Testing biological fluids and tissues for a variety of conditions, diseases, markers and drugs is an integral part of health care. A blood panel, for example, is standard protocol in an annual physical examination. The analysis of saliva is less routine but can be extremely helpful in various situations. For example, saliva can be screened to help identify the presence of Cushing's disease, anovulation, HIV, cancer, parasites, hypogonadism and allergies, and for the presence of various compounds. Saliva can also be tested for tooth and gum health, and oral cavity cleanliness.

One of the barriers to testing biological fluids such as saliva is the availability of the requisite analysis tools. By way of example, it would be desirable for dentist offices to routinely offer saliva testing for monitoring oral health, but saliva testing instruments are beyond the reach for many practices. Likewise, the ordinary health-conscious consumer does not have access to the instrumentation necessary to monitor their oral health.

Another challenge associated with saliva analysis is specimen instability. Saliva is replete with microbes and proteolytic enzymes which alter the constituents of specimens over time, plus various biomarkers are affected by pH and temperature. For these and many other reasons, collecting and storing saliva specimens for later analysis is not simply a matter of spitting in a cup and testing it later.

As can be seen, there is a need for a saliva testing system and method that overcomes the shortcomings of the prior art. It is desirable that this system and method can facilitate sampling saliva in a non-clinical setting by non-trained personnel, stabilize that specimen for subsequent screening, analyze the specimen for various markers of oral and general health, and report those results. It is desirable that this system and method are reasonably easy to use, effectively stabilize saliva specimens, and yield reproducible and accurate screening data.

### SUMMARY OF THE INVENTION

A saliva testing system facilitates obtaining a saliva sample and preparing a stabilized saliva test sample for subsequent analysis. The system preferably includes a kit having various collection and preparation implements, additives such as purified water and stabilizer, means for mailing the sample, and instructions for use. A user obtains a saliva sample, prepares the stabilized saliva test sample as instructed, and mails the sample to a 3^{rd} party for analysis. The 3^{rd} party tests the sample for tooth and gum health indicators using known testing instrumentation, and sends the user a report summarizing their tooth and gum health. The preferred stabilizer is 1× TE pH 8.0.

### BRIEF DESCRIPTION OF THE DRAWINGS

FIG. 1 schematically depicts an overview of the system and method;

FIG. 2 schematically depicts some steps related to specimen collection;

FIG. 3 schematically depicts some steps related to specimen preparation; and

FIG. 4 schematically depicts some steps related to specimen handling and analysis.

### DETAILED DESCRIPTION OF THE INVENTION

As used herein, the following structure numbers shall refer to the various structures of the invention as depicted in the figures:
10—Saliva testing system;
20—Test subject;
22—Saliva;
24—Saliva specimen;
30—Testing kit;
31—Transfer pipette;
32—Collection cup;
33—Swishing fluid;
34—Tracking card;
35—Test strip;
36—Reactive pad;
37—First specimen tube;
38—Second specimen tube;
39—Shipping pouch;
40—Stabilizer A;
41—Stabilizer B;
42—First stabilized specimen;
43—Second stabilized specimen;
50—Saliva testing instrument;
52—Sample diluent;
54—Assay strip; and
56—Test report.

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, the present invention is a system and method of testing stabilized saliva whereby a user, typically a test subject, obtains a specimen of saliva, stabilizes it, mails it to a testing facility for analysis and receives a report of the results.

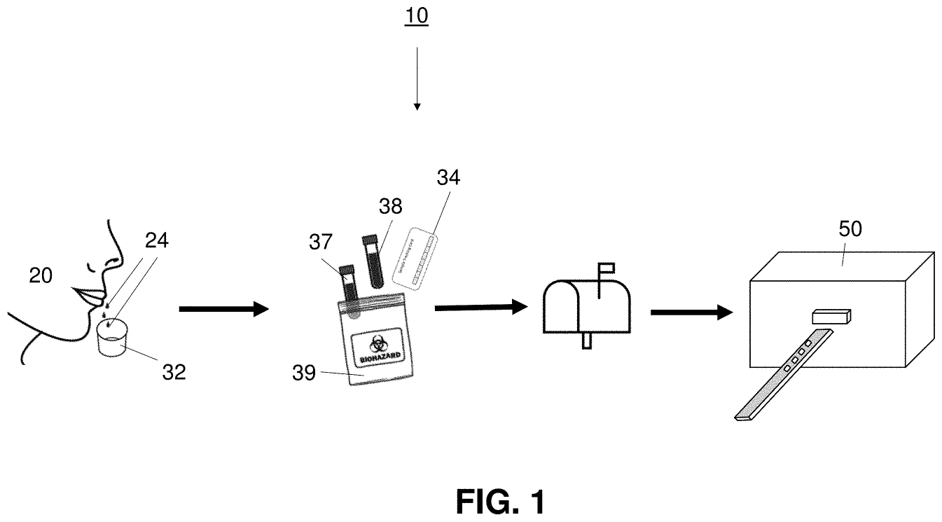

FIG. 1 depicts an overview of the system and method with test subject 20 orally expressing saliva specimen 24 into collection cup 32; inserting first and second specimen tubes 37, 38, and tracking card 34 into shipping pouch 39; and mailing shipping pouch 39 to a testing facility for analysis by saliva testing instrument 50.

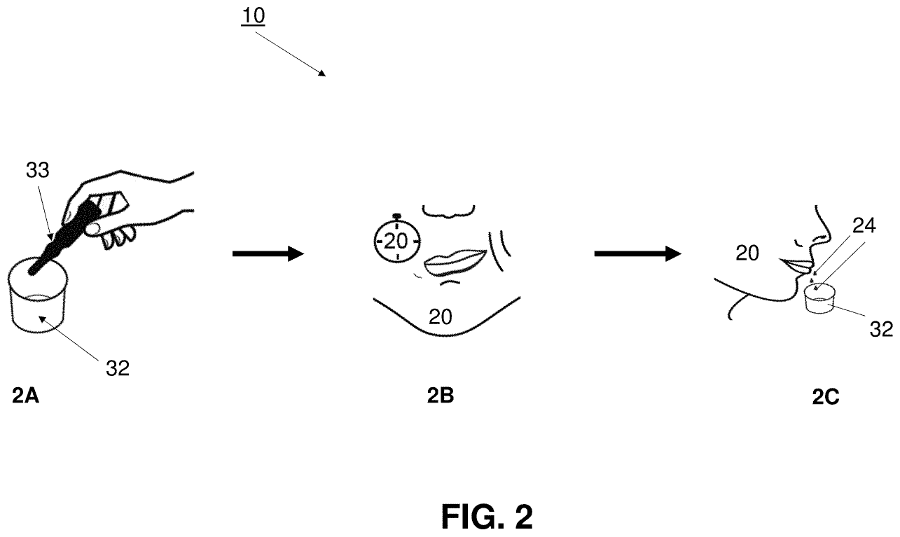

FIG. 2 depicts some specimen collection steps in saliva testing system 10. Referring to FIG. 2A, swishing fluid 33 is introduced into empty collection cup 32. In a preferred embodiment swishing fluid 33 is approximately 6 mL of sterile/purified water, preferably split between two 3 mL vessels. As shown in FIG. 2B, test subject 20 swishes swishing fluid 33 in their mouth for approximately 20 seconds. Saliva specimen 24, which is a mixture of saliva 22 (not shown) and swishing fluid 33, is then expelled into collection cup 32, as shown in FIG. 2C. It is preferred that the saliva specimen is collected when the test subject wakes up in the morning, before brushing teeth or consuming food or drink in order to obtain a baseline of a subject's natural bacteria load and type. If this is not possible it is preferred that the test subject not brush or floss 30 minutes prior to collection and that they avoid food one hour prior to collection.

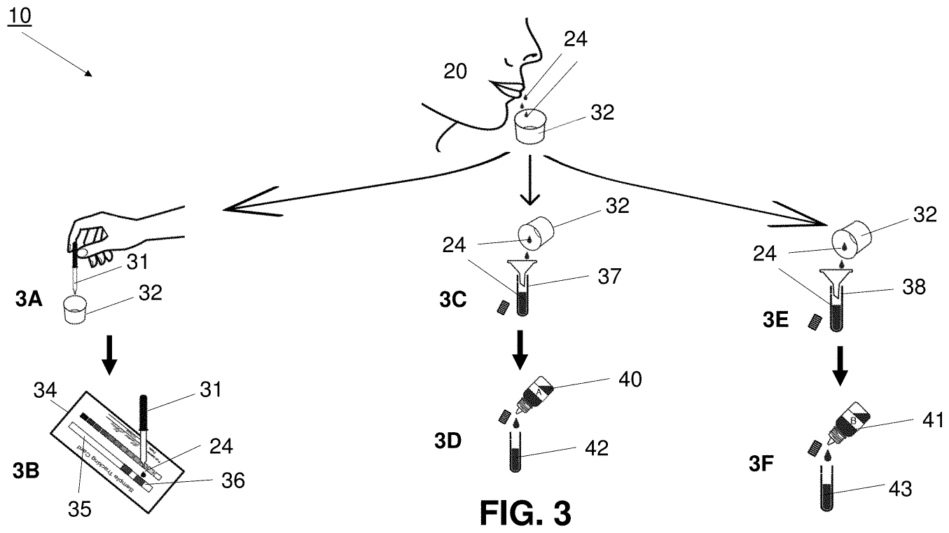

FIG. 3 depicts some specimen preparation steps using saliva specimen 24 in collection cup 32 which has been collected as described in FIG. 2C. Saliva specimen 24 is preferably allocated for use in multiple analyses, with FIG. 3 depicting a three-way division having a first allocation for deposition onto a tracking card, a second allocation for testing a specific set of biomarkers, and a third allocation for testing a different set of biomarkers. It should be understood that varying numbers of allocations are within the scope of this invention.

In FIG. 3A an aliquot of saliva specimen 24 is removed from collection cup 32. In FIG. 3B that aliquot is deposited onto reactive pad 36 of test strip 35 on tracking card 34. Tracking card 34 preferably tests for pH and resistance to acid on the freshly collected sample. In a preferred embodiment tracking card 34 is photographed for subsequent submission to the testing facility, for example via a HIPAA-compliant text messaging system.

FIGS. 3C and 3D depict dispensing saliva specimen 24 from collection cup 32 up to a provided fill line (not shown) on first specimen tube 37 and adding stabilizer A 40 to yield first stabilized specimen 42. Although FIG. 3C depicts pouring saliva specimen 24 into first specimen tube 37 it is preferable to use a dropper such as transfer pipette 31 given it is generally more accurate to reach a fill line by dropping versus pouring. In a preferred embodiment first stabilized specimen 42 consists of approximately 50% stabilizer A and approximately 50% saliva specimen 24 by volume. Although not shown, first specimen tube 37 is preferably capped then inverted approximately 10 times to mix. In a preferred embodiment first stabilized specimen 42 is subsequently tested for cavity-causing bacteria, an indicator of tooth health.

FIGS. 3E and 3F depict dispensing saliva specimen 24 from collection cup 32 into second specimen tube 38 and adding stabilizer B 41 to yield second stabilized specimen 43. Although not shown, for the reasons discussed above with respect to FIG. 3C it is preferable to employ transfer pipette 31 to transfer saliva specimen up to fill line on specimen tube. In a preferred embodiment second stabilized specimen 43 consists of approximately 10% stabilizer and 90% saliva specimen 24 by volume. Second specimen tube 38 is preferably capped then inverted approximately 10 times to mix. In a preferred embodiment second stabilized specimen 43 is subsequently tested for inflammatory factor 1—red blood cells; inflammatory factor 2—white blood cells; and protein biomarkers, all of which are indicative of gum health.

It should be understood that stabilizer A 40 and stabilizer B 41 can be chemically identical although their proportional volume will likely vary in first stabilized specimen 42 versus second stabilized specimen 43. By way of example, in a preferred embodiment first stabilized specimen 42 consists of 1 mL of stabilizer plus 1 mL of saliva specimen, thereby yielding a mixture of 50% stabilizer, while second stabilized specimen 43 consists of 300 μL of stabilizer plus 3 mL of saliva specimen, thereby yielding a mixture of 10% stabilizer.

In a preferred embodiment stabilizers A and B 40, 41 are a TE buffer, with 1× TE pH 8.0 being most preferred. Among other sources this solution is commercially available as MB-040 from Rockland Immunochemicals.

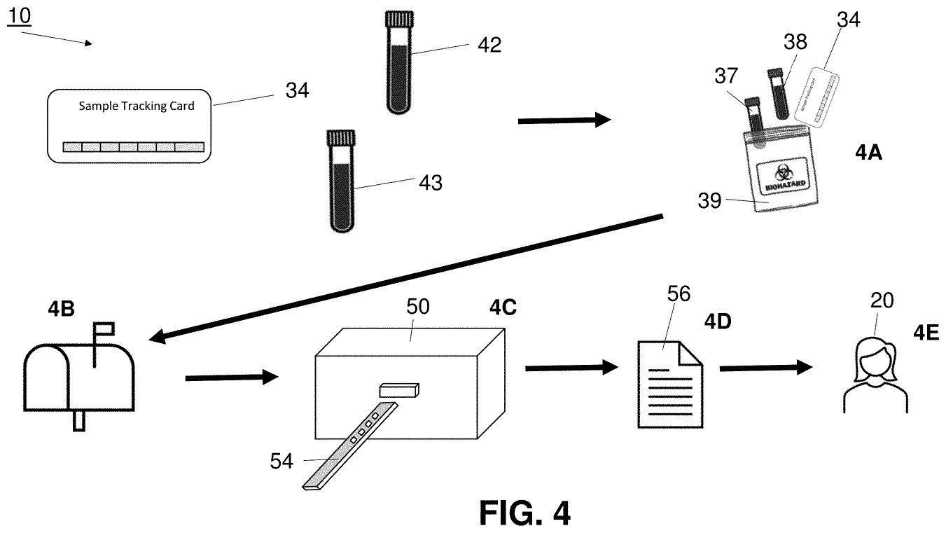

Referring to FIG. 4, tracking card 34, first stabilized specimen 42 and second stabilized specimen 43, all prepared in accordance with FIG. 3, are put into shipping pouch 39, which may be configured and labeled as a “BIOHAZARD” container. This is depicted in FIG. 4A. Shipping pouch 39 is mailed (FIG. 4B) to a testing facility having saliva testing instrument 50. In a preferred embodiment first and second stabilized specimens 42, 43 are loaded onto assay strip 54 and tested in accordance with standard testing protocols, as shown in FIG. 4C. Test report 56 is generated from said testing (FIG. 4D) and sent to test subject 20 and/or their designated agent. In a preferred embodiment saliva testing instrument 50 is modified and validated as a laboratory-developed test, with a commercially available but unmodified example being the SillHa Salivary Testing Instrument from ARKRAY USA, Inc., in Minneapolis, Minn.

In using a preferred embodiment of the present invention test subject 20 is provided the tools and information to collect and prepare their specimen without special training or equipment. This can be accomplished by providing testing kit 30 (not shown) including transfer pipette 31, collection cup 32, 2 vessels of 3 mL swishing fluid 33, tracking card 34, first specimen tube 37, second specimen tube 38, shipping pouch 39, stabilizer A 40, stabilizer B 41 and instructions for use. It is desirable that swishing fluid 33, stabilizer A 40 and stabilizer B 41 are pre-measured so test subject 20 simply empties the contents of the associated vessel, for example “Stabilizer A” or “Stabilizer B” into the corresponding specimen tube 37, 38, without needing to measure specific volumes. As described above stabilizers A and B 40, 41 may be the same chemical composition, although their associated vessels would likely contain different volumes.

The present invention is based on observations and data that indicate markers in unstabilized saliva change more than markers in stabilized saliva over time.

Ten oral rinse samples obtained at the same time from the same source were analyzed for five different markers, with two sample, A and B, per marker. They were analyzed at 0, 2, 4 and 6 hours using the SillHa Salivary Testing Instrument model LH-4912 in accordance with the associated standard protocol. The instrument measures oral rinse samples using the dual-wavelength reflectance method wherein the change in color of the test strip after sample application is measured by the reflectance of light with wavelengths of 565 nm, 635 nm and 760 nm. Results pertain to levels of cariogenic bacteria, acidity, buffer capacity, leukocyte, protein and ammonia, and are reported numerically as the Raw Value (0-100) and categorized as “low”, “average” or “high” based on the following numerical ranges:

| | LOW | AVERAGE | HIGH |
|---|---|---|---|
| Cavity-causing | 0-24 | 25-47 | 48-100 |
| bacteria | | | |
| Inflammatory | 0-14 | 15-29 | 30-100 |
| Factor 1 - RBC | | | |
| Inflammatory | 0-37 | 38-60 | 61-100 |
| Factor 2 - WBC | | | |
| Protein | 0-36 | 37-53 | 64-100 |
| Bacterial | 0-43 | 44-63 | 64-100 |
| Byproduct | | | |

Stabilized samples employ a different scale for “low”, “average” or “high” based on the SillHa Raw Value. These ranges are set forth below:

| | STABILIZED | STABILIZED | STABILIZED |
|---|---|---|---|
| | LOW | AVERAGE | HIGH |
| Cavity-causing | 0-37 | 38-63 | 64-100 |
| bacteria | | | |
| Inflammatory | 0-24 | 25-76 | 77-100 |
| Factor 1 - RBC | | | |
| Inflammatory | 0-42 | 43-58 | 59-100 |
| Factor 2 - WBC | | | |
| Protein | 0-42 | 43-58 | 59-100 |
| Bacterial | 0-38 | 39-62 | 63-100 |
| Byproduct | | | |

Table 1 depicts unstabilized Raw Values and associated categories, along with notations where the sample changed at least one category, or example from low to high, over the testing period:

TABLE 1
Saliva samples without stabilizer

| | Sample | | | | | Description of |
|---|---|---|---|---|---|---|
| Marker | ID | 0 | 2 hr | 4 hr | 6 hr | Changes |
| Cavity-causing | A | 0, low | 4, low | 0, low | 0, low | |
| bacteria | B | 0, low | 0, low | 0, low | 65, high | increase +2 |
| | | | | | | category change |
| Inflammatory | A | 21, Ave | 25, Ave | 14, Ave | 33, high | increase +1 |
| | | | | | | category change |
| Factor 1 - RBC | B | 13, low | 9, low | 10, low | 27, Ave | increase +1 |
| | | | | | | category change |
| Inflammatory | A | 55, Ave | 37, Ave | 27, Low | 53, Ave | |
| Factor 2 - WBC | B | 50, Ave | 27, Low | 22, Low | 52, Ave | |
| Protein | A | 47, Ave | 60, high | 36, Ave | 71, high | increase +1 |
| | | | | | | category change |
| | B | 39, Ave | 20, low | 48, Ave | 57, high | increase +1 |
| | | | | | | category change |
| Bacterial | A | 77, high | 96, high | 72, high | 95, high | |
| Byproduct | B | 60, ave | 45, ave | 71, high | 90, high | increase +1 |
| | | | | | | category change |

It is noted that 60% of the samples demonstrated at least 1 category change over the 6-hour time period.

TABLE 2
Summary of saliva samples without stabilizer

| | Total # of | % |
|---|---|---|
| Total # | category changes | changes |
| samples | at last time point | in dataset |
| 10 | 6 | 60% |

Table 3 reports the effect of time on the five markers in unstabilized samples over the course of five days using the same protocol.

TABLE 3
Saliva samples without stabi izer

| | Sample | | | | | | Description of |
|---|---|---|---|---|---|---|---|
| Marker | ID | 0 | day 2 | day 3 | day 4 | day 5 | Changes |
| Cavity-causing | A | 25, low | 22, low | 59, high | 73, high | 43 ave | increase +1 |
| bacteria | | | | | | | category |
| | | | | | | | change |
| | B | 0, low | 20, low | 37, ave | 33, ave | 22 low | |
| | C | 46, ave | 0, low | 60. high | 51, high | 41 ave | |
| Inflammatory | A | 28, ave | 1, low | 15, ave | 16, ave | 3 low | decrease -1 |
| Factor 1 - RBC | | | | | | | category |
| | | | | | | | change |
| | B | 6, low | 6, low | 5, low | 7, low | 9 low | |
| | C | 18, ave | 11, low | 16, ave | 19, ave | 16 ave | |
| Inflammatory | A | 66, high | 64, high | 71, high | 80, high | 38 ave | decrease -1 |
| Factor 2 - WBC | | | | | | | category |
| | | | | | | | change |
| | B | 4, low | 1, low | 8, low | 14, low | 17 low | |
| | C | 44, ave | 33, low | 65, high | 66, high | 75 high | increase +1 |
| | | | | | | | category |
| | | | | | | | change |
| Protein | A | 52, ave | 41, ave | 56, high | 58, high | 28 low | decrease -1 |
| | | | | | | | category |
| | | | | | | | change |
| | B | 22, low | 19, low | 20, low | 18, low | 16 low | |
| | C | 44, ave | 46, ave | 51, ave | 55, high | 46 ave | |
| Bacterial | A | 70, high | 100, high | 99, high | 100, high | 100 high | |
| Byproduct | B | 49, ave | 58, ave | 81, high | 69, high | 83 high | increase +1 |
| | | | | | | | category |
| | | | | | | | change |
| | C | 88, high | 100, high | 96, high | 100, high | 98 high | |

TABLE 4
Summary of saliva samples without stabilizer

| | Total # of | % |
|---|---|---|
| Total # | category changes | changes |
| samples | at last time point | in dataset |
| 15 | 6 | 40% |

As shown, 40% of the samples demonstrated at least 1 category change over the 6-day time period. It is notable that the 60% and 40% changes over 6 hours and 5 days, respectively, are not inconsistent. Rather, some data will curve over time, for example bacteria may initially increase in number then decline as nutrition is consumed, which would exhibit a curved versus linear growth pattern.

Table 5 reports the effect of time on the five markers in stabilized samples over the course of five days using the same protocol.

TABLE 5
Saliva samples with stabilizer

| | Sample | | | Description of |
|---|---|---|---|---|
| Marker | ID | Day 1 | DAY 5 | Changes |
| Cavity-causing | A | 22 low | 27 low | |
| bacteria | B | 22 low | 8 low | |
| | C | 22 low | 17 low | |
| | D | 46 ave | 59 ave | |
| | E | 0 low | 22 low | |
| | F | 8 low | 33 low | |
| | G | 37 ave | 35 ave | |
| Inflammatory | A | 23 ave | 14 ave | |
| Factor 1 - RBC | B | 100 high | 71 high | |
| | C | 6 low | 7 low | |
| | D | 35 high | 29 high | |
| | E | 37 high | 33 high | |
| | F | 8 low | 8 low | |
| | G | 14 ave | 16 ave | |
| Inflammatory | A | 58 ave | 52 ave | |
| Factor 2 - WBC | B | 53 ave | 68 ave | |
| | C | 28 low | 49 low | |
| | D | 80 high | 89 high | |
| | E | 42 ave | 61 ave | |
| | F | 43 ave | 60 ave | |
| | G | 29 low | 53 ave | increase +1 |
| | | | | category change |
| Protein | A | 52 ave | 28 low | decrease −1 |
| | | | | category change |
| | B | 61 high | 49 ave | decrease −1 |
| | | | | category change |
| | C | 30 low | 32 low | |
| | D | 63 high | 60 high | |
| | E | 58 high | 59 high | |
| | F | 18 low | 20 low | |
| | G | 21 low | 14 low | |
| Bacterial | A | 43 ave | 60 high | increase +1 |
| Byproduct | | | | category change |
| | B | 81 high | 94 high | |
| | C | 52 ave | 60 high | increase +1 |
| | | | | category change |
| | D | 91 high | 100 high | |
| | E | 71 high | 84 high | |
| | F | 41 low | 24 low | |
| | G | 25 low | 13 low | |

14% of the samples demonstrated at least 1 category change over the 6-day time period. These data suggest that saliva samples with stabilizer of the present invention are significantly more stable than unstabilized samples.

TABLE 6
Summary of saliva samples with stabilizer

| | Total # of | |
|---|---|---|
| Total # | category changes | % changes in |
| samples | at last time point | dataset |
| 35 | 5 | 14% |

It should be understood that the foregoing relates to exemplary embodiments of the invention and that modifications may be made without departing from the spirit and scope of the invention as set forth in the following claims. Examples of modifications include the inclusion of additional markers and/or a totally “at home” system that does not require sending out a sample. Terms such as “substantially” and the like shall mean within reasonable bounds when considering limitations such as machines, materials, manufacturing methods, and people. By way of example, a “substantially smooth” surface means there are no intentional bumps or irregularities. All ranges set forth herein include the endpoints as well as all increments there between, even if not specifically stated. By way of example 1 to 2 inches includes 1 inch, 1.000001 inches and so forth. Finally, unless otherwise stated or contrary to common sense, “approximate” and the like shall mean +/−10%.

What is claimed is:

1. A stabilizer for preparing a saliva test sample, said stabilizer comprised of TE buffer, wherein said saliva test sample is suitable for analysis of at least one indicator selected from the group consisting of tooth health and gum health.

2. The stabilizer of claim 1 wherein said tooth health indicator is the presence of cavity-causing bacteria.

3. The stabilizer of claim 1 wherein said gum health indicator is at least one marker selected from the group consisting of inflammatory factors and protein biomarkers.

4. The stabilizer of claim 3 wherein said inflammatory factors are selected from the group consisting of red blood cell markers and white blood cell markers.

5. The stabilizer of claim 1 wherein said saliva test sample includes purified water.

6. The stabilizer of claim 1 wherein said TE buffer is 1× TE pH 8.0.

7. The stabilizer of claim 6 wherein the saliva test sample includes approximately 10% to approximately 50% TE buffer by volume.

8. A stabilized saliva specimen including:
a. A saliva specimen comprising a mixture of saliva and purified water; and
b. TE buffer.

9. The stabilized saliva specimen of claim 8 wherein said TE buffer is 1× TE pH 8.0.

10. The stabilized saliva specimen of claim 9 wherein the ratio of said saliva specimen to said TE buffer is approximately 1:1 by volume.

11. The stabilized saliva specimen of claim 9 wherein the ratio of said saliva specimen to said TE buffer is approximately 9:1 by volume.

12. A method of testing saliva comprising the steps of:
a. Providing a kit to a test subject, said kit containing a collection cup, a volume of swishing fluid, a tracking card, a first specimen tube, a second specimen tube, a shipping pouch, a volume of stabilizer A and a volume of stabilizer B;
b. Providing instructions to said test subject, said instructions including the directive to send stabilized saliva specimens to a testing facility;
c. Testing said stabilized saliva specimens at said testing facility, and
d. Providing a test report to said test subject.

13. The method of testing saliva according to claim 13 wherein said volume of stabilizer A is a TE buffer.

14. The method of testing saliva according to claim 13 wherein said TE buffer is 1× TE pH 8.0.

15. The method of testing saliva according to claim 12 wherein said step of providing instructions to said test subject includes the directive to obtain a saliva specimen in the morning before brushing teeth.

16. The method of testing saliva according to claim 12 further including the step of providing feedback on said test report to said test subject.
